(19) United States
(12) Patent Application Publication
(10) Pub. No.: US 2022/0240789 A1
(43) Pub. Date: Aug. 4, 2022

(54) ESTIMATION APPARATUS, METHOD AND PROGRAM

(71) Applicant: NEC Corporation, Minato-ku, Tokyo (JP)

(72) Inventors: Utkarsh SHARMA, Tokyo (JP); Yoshifumi ONISHI, Tokyo (JP)

(73) Assignee: NEC Corporation, Minato-ku, Tokyo (JP)

(21) Appl. No.: 17/424,823

(22) PCT Filed: Feb. 1, 2021
(86) PCT No.: PCT/JP2019/003691

§ 371 Date: Jul. 21, 2021

(51) Int. Cl. A61B 5/024; G06V 10/40; G06T 7/90; G06V 10/25; G06T 7/00; A61B 5/00

(52) U.S. Cl. CPC A61B 5/024; G06V 10/40; G06T 7/90; G06V 10/25; G06T 7/0012; G06T 2207/30201; A61B 5/7267; A61B 5/7203; A61B 5/7253; A61B 5/7225; G06T 2207/20081; A61B 5/7221

(57) ABSTRACT

An estimation apparatus (30) includes a first estimation unit (31) configured to estimate a first pulse rate from a first video data which is captured a body part and output a first feature data derived based on the first video data; a training unit (32) configured to train a determination model to determine a confidence value which indicates an reliability in the estimation of the first pulse rate and a physiological information measured from the body; an acquiring unit (33) configured to acquire a second feature data derived by the first estimation unit, and acquire the confidence value of the second pulse rate using the second feature data and the determination model trained by the training unit; and a second estimation unit (34) configured to estimate a third pulse rate based on the acquired confidence value.

### TECHNICAL FIELD

The present disclosure relates to an estimation apparatus, method and program. In particular, the present disclosure relates to an estimation apparatus, method and program to estimate pulse rate.

### BACKGROUND ART

There's been growing interest in measurement of physiological information aimed at stress detection, health care, and accident prevention. Heart rate (HR) measurements are especially important as it's been shown that human psychological states as stress, arousal, and sleepiness can be estimated from the HR. While the HRs are usually measured by contact-based means, especially electrocardiography, however for the aforementioned applications, continuous, simpler measurement is necessary. To this end, in recent years, HR measurement techniques employing videos captured with commonly-used cameras have been proposed.

Several techniques to estimate HR (or other quasi-periodic physiological signals) from a sequence of pictures of the human face (or any other body part where a skin is exposed) have been studied. For example, Non-Patent Literature 1 (NPL1) discloses a technique to estimate HR from face videos. The NPL1 uses amplitude fluctuations (due to cardiac activity) in the green-color channel of a color video of human face to estimate heart rate in the presence of noise introduced by head motion and/or facial expressions.

Further, Patent literature 1 (PTL1) discloses a technique to estimate pulse rate from videos based on the idea that some small regions on the face contain stable pulse wave information, which can be derived by choosing only those areas on face, which have low variability in skin color over time. The main steps of the PTL1 include:

a) generate facial feature points by tracking face on video;

b) divide the area under observation (also called the region of interest (ROI)) into smaller parts, known as sub-ROIs;

c) extract pulse signal (green channel amplitude fluctuations) from each sub-ROI;

d) create dynamic ROI filter to select “reliable” sub-regions, i.e., give large weight to sub-ROIs with low local (temporal) variance of pulse signal (known as “trusted region”);

e) combine (using weights assigned in step (d)) pulse wave information of the trusted regions to get a single time-series of pulse wave;

f) perform frequency analysis and estimate final pulse signal from the pulse wave information of only “trusted regions”.

Furthermore, Patent literature 2 (PTL2) and Patent literature 3 (PTL3) disclose techniques to extract periodic component from multi-component pulse signals. The basic steps involved in the PTL2 and PTL3 include:

a) pre-select a reference periodicity value (using pre-existing knowledge of data, or rough estimate of periodicity of long-term pulse signal—for example, calculating mean periodicity/frequency value of the pulse signal over a long time, ranging from 1 minute to few hours);

b) find a reference pulse signal “cyclet”, also known as a “repeat base unit” (one cycle of pulse signal where periodicity is same as/close to reference periodicity or auto-correlation is maximized);

c) From the multicomponent pulse-signal, extract cyclets (or periodic components) which have high correlation (above a threshold value) with the repeat base unit, or alternatively, a periodicity close to (within a threshold value) the reference periodicity.

### CITATION LIST

### Patent Literature

PTL 1: Japanese Unexamined Patent Application Publication No. 2018-164587
PTL 2: U.S. Pat. No. 6,262,943 B1
PTL 3: U.S. Pat. No. 5,584,295 A

### Non Patent Literature

NPL 1: Sharma, Umematsu, Tsujikawa, and Onishi, “Adaptive Heart Rate Estimation From Face Videos”, IEICE SBRA 2018

### SUMMARY OF INVENTION

### Technical Problem

However, each of the above mentioned techniques has a problem of deterioration of estimation accuracy. The reason for the occurrence of the problem is that several uncontrollable noise sources, which includes rigid and non-rigid motion performed by the person under observation, occlusion, face tracking error, light source changes, and the like, introduce complex corruption to observed signal and make it difficult to estimate HR.

The present disclosure has been accomplished to solve the above problems and an object of the present disclosure is thus to provide an estimation apparatus, method and program to improve the estimation accuracy.

### Solution to Problem

An estimation apparatus according to a first exemplary aspect of the present disclosure includes a first estimation unit configured to estimate a first pulse rate from a first video data which is captured a body part where a skin is exposed and output a first feature data derived based on the first video data in order to estimate the first pulse rate; a training unit configured to train a determination model to determine a confidence value which indicates an reliability in the estimation of the first pulse rate based on the first feature data and a physiological information measured from the body; an acquiring unit configured to acquire a second feature data derived by the first estimation unit when the first estimation unit estimates a second pulse rate from a second video data which is captured the body part to be estimated, and acquire the confidence value of the second pulse rate using the second feature data and the determination model trained by the training unit; and a second estimation unit configured to estimate a third pulse rate based on the acquired confidence value.

An estimation method according to a second exemplary aspect of the present disclosure includes performing a first estimation process to estimate a first pulse rate from a first video data which is captured a body part where a skin is exposed; outputting a first feature data derived based on the first video data in the first estimation process; training a determination model to determine a confidence value which indicates a reliability in the estimation of the first pulse rate based on the first feature data and a physiological information measured from the body; performing a second estimation process to estimate a second pulse rate from a second video data which is captured the body part to be estimated; outputting a second feature data derived based on the second video data in the second estimation process; acquiring the confidence value of the second pulse rate using the second feature data and the trained determination model; and estimating a third pulse rate based on the acquired confidence value.

A non-transitory computer readable medium storing an estimation program according to a third exemplary aspect of the present disclosure, the program causes a computer to execute: a first estimation process for estimating a first pulse rate from a first video data which is captured a body part where a skin is exposed; a process for outputting a first feature data derived based on the first video data in the first estimation process; a process for training a determination model to determine a confidence value which indicates a reliability in the estimation of the first pulse rate based on the first feature data and a physiological information measured from the body; a second estimation process for estimating a second pulse rate from a second video data which is captured the body part to be estimated; a process for outputting a second feature data derived based on the second video data in the second estimation process; a process for acquiring the confidence value of the second pulse rate using the second feature data and the trained determination model; and a process for estimating a third pulse rate based on the acquired confidence value.

### Advantageous Effects of Invention

According to the exemplary aspects of the present disclosure, it is possible to provide an estimation apparatus, method and program to improve the estimation accuracy.

### BRIEF DESCRIPTION OF DRAWINGS

FIG. 1 is a block diagram illustrating a structure of a pulse rate estimation apparatus according to a first exemplary embodiment of the present disclosure.

FIG. 2 is a flowchart for explaining a training stage for pulse rate estimation according to the first exemplary embodiment of the present disclosure.

FIG. 3 is a flowchart for explaining an initial pulse rate estimation processing according to the first exemplary embodiment of the present disclosure.

FIG. 4 is a flowchart for explaining a training process of determination model according to the first exemplary embodiment of the present disclosure.

FIG. 5 is a diagram for explaining a concept of a process to estimate an initial pulse rate from captured images.

FIG. 6 is a diagram for explaining a concept of a process to train the determination model from feature data.

FIG. 7 is a flowchart for explaining the testing stage for pulse rate estimation according to the first exemplary embodiment of the present disclosure.

FIG. 8 is a diagram for explaining a concept of a process to estimate a final pulse rate from an initial pulse rate at the testing state

FIG. 9 is a block diagram illustrating a hardware structure of the pulse rate estimation apparatus according to the first exemplary embodiment of the present disclosure.

FIG. 10 is a block diagram illustrating a structure of an estimation apparatus according to a second exemplary embodiment of the present disclosure.

FIG. 11 is a flowchart for explaining an estimation method according to the second exemplary embodiment of the present disclosure.

### DESCRIPTION OF EMBODIMENTS

Hereinafter, specific embodiments to which the present disclosure including the above-described example aspects is applied will be described in detail with reference to the drawings. In the drawings, the same elements are denoted by the same reference signs, and repeated descriptions are omitted for clarity of the description.

The problem to be solved by the present disclosure is explained below in detail. In real world scenarios noise, from background illumination changes, rigid head motion and/or change in facial expressions, partial occlusion as well as noise introduced due to inaccurate face tracking, often affects performance of pulse extraction. Due to a variety of noises, the pulse signal can get corrupted and the expected pulse fluctuations can't be captured effectively, and as a result the performance (e.g. accuracy) of pulse rate estimation methods is negatively affected.

Further, the NPL 1 employs head motion/facial expression detection as a method for emphasizing ROI sub-regions on the face that have been least corrupted by noise from head motion and/or facial expression. This is followed by noise-specific correction steps which try to remove the noisy fluctuations from pulse signal. However, in practice, the NPL 1 can only partially remove the noisy fluctuations because of the following reasons:

i. The above mentioned noise-specific correction steps can't remove the noise that is generated by factors other than head motion and facial expressions; (for example) some noise is always generated because the face tracking is inaccurate and the obtained pulse signals are corrupted by tracking error noise, while there is also noise from factors like ambient light changes, or some very hard-to-remove noise like partial occlusion of face region, or large head motion and facial expression at the same time (for example, during intense laughing).

ii. Some video frames which do not have head motion or facial expression have noise from other sources. Therefore these video frames should not ideally be considered “reliable”. As a result, the NPL 1 considers many noisy HR estimates as “reliable”. Accordingly other neighboring estimates are inaccurate because spectral peak tracking is trying to find HR only in a small range close to reliable HR value.

iii Some video frames which have head motion or facial expression can be considered “reliable”. Because the noise correction steps accurately removed the noise caused by head motion/facial expression. But the NPL 1 considers all such frames as “unreliable”.

Basically, although presence of head motion/facial expression is correlated to the noise in pulse signal but that is neither necessary, nor sufficient condition to consider the estimated HR as “unreliable”. There are many other factors that introduce noise in the pulse signal which go undetected in the NPL 1.

This shows us that not all sources of noise can be considered treatable or controllable, especially in a real situation, where there is no restriction on the actions that the person under observation can perform. Past studies in the field of video-based physiological signal extraction also conclude that some noise which is present in the pulse signals (color fluctuations on face) are not separable from the cardiac fluctuations, in which case, it becomes extremely difficult to estimate accurate HR. To solve this problem, we need a decision making system that can accurately detect video frames which have noise, regardless of the source of the noise or the conditions in which the noise was generated. If we are able to correctly detect these “unreliable” scenarios, the problems of prior art, mentioned in points i, ii and iii above, will be solved. Furthermore, if we are able to get a quantitative, and not just qualitative, measure for the reliability (reliability index or confidence value) of pulse signals and the HR estimated from them, we can: a) tell the patients how confident we are about the estimated HR; b) improve the accuracy of low reliability HR estimates by using neighboring high reliability HR estimates as reference.

Further, it is important to note that the “trusted region” selection in PTL 1 is akin to the dynamic ROI filter of the NPL1, and every shortcoming of the dynamic ROI selection (using the dynamic ROI filter) process of the NPL1 also applies to the “trusted region” selection process of PTL 1. One of differences between the PTL 1 and the present disclosure is that the PTL 1 only tries to select reliable areas on face. In other words, in the PTL 1, only spatial reliability is achieved. However, the spatial reliability is not enough to get accurate heart rate information in the presence of noise from external and internal sources. The biggest reason for this is that spatial reliability of the PTL 1 is only relative, not absolute. That is, the “trusted regions” discussed in the PTL 1 are only more trusted than other regions, but their reliability in absolute terms is not determined. So, when the noise is large (shaking of head, a big laugh, tracking failure, etc.), or when the entire face is affected by a noise (light source changes, tracking failure, etc.), the PTL 1 only tries to determine which reason is least affected by this noise. However, since all regions on the face contain large noise, the PTL 1 fails to select regions which are actually “trusted”, resulting in poor pulse rate estimation accuracy. For dealing with cases of large noise or noise affecting entire face, we need to have spatial as well as temporal reliability determination. The present disclosure achieves this requirement by identifying (relative) spatial reliability in the ROI selection & pulse extraction unit 104 to be described later as well as absolute temporal reliability determination using the confidence level estimation process performed in the confidence regressor 107 and the confidence estimator 108 units to be described later. The confidence level estimate achieved in the confidence estimator 108 is actually a measure of temporal reliability of the video input data, and helps in the identification on cases of large noise or noise affecting entire face, which leads to better noise removal and more accurate pulse rate estimation than in the PTL 1.

In prior arts PTL 2 and PTL 3, the extracted periodic component is heavily biased towards the reference periodicity value, which may or may not be “reliable”.

i. If the reference periodicity value is calculated over a long time (>2 minutes), the prior arts will miss minor changes in the periodicity over time, due to being biased towards reference periodicity value, which will lead to low accuracy in pulse rate (heart rate) estimation.

ii. On the other hand, if the reference periodicity is calculated over a short time (10-30 seconds), it is prone to being inaccurate in scenarios where noise is present over a few seconds (talking, laughing, nodding, etc.) or face tracking failures over a few seconds.

Furthermore, the reference periodicity value (or repeat base unit) tends to become less “reliable” when we move away from it in time, since pulse rate is not constant over time and is likely to keep changing over time. So, the idea of using a reference HR is only reliable for very short times (2-4 seconds). So, in order to make the best use of a reference periodicity signal while not being heavily biased towards the reference, we need a method that continuously adapts the periodicity/pulse rate search range depending on:

a) the time elapsed since the reference periodicity (or repeat base unit) was selected;

b) the reliability index (or confidence level) of the reference periodicity value (or reference pulse rate or repeat base unit) itself.

We need a method to continuously determine (temporal) reliability index/confidence level for each estimated periodicity/pulse rate value and use confidence levels or neighboring video frames, so as to adaptively extract the reference pulse rate value and the resulting periodic component. In this way, due to having a quantitative reliability index, we will be able to extract relatively more reliable estimates from a noisy set of video frames by adapting the threshold values and reference periodicity (for periodic component extraction) while taking into consideration (instead of completely neglecting) the noisy estimates with low/medium reliability indices and not being too biased towards estimates with high reliability index.

Further, the present disclosure may train a neural network to learn the confidence levels for a big dataset of multi-component pulse signal segments (extracted from video of a tracked face as in the PTL 1). This trained neural network is then used on test data to extract reliability index of each new pulse signal segment (or “cyclet”), followed by adaptive periodic component extraction that uses reliability indices of recent past (last 2-10 seconds) pulse rate estimates as well as current pulse rate reliability to dynamically select reference pulse rate value and extract a periodic component from the multi-component pulse signal.

Based on the above, the first problem of HR estimation in prior arts is the deterioration of estimation accuracy. The reason for the occurrence of the first problem is that several uncontrollable noise sources introduce complex corruption to observed signal and make it difficult to estimate HR. Note that, several uncontrollable noise sources include, for example, rigid and non-rigid motion performed by the person under observation, occlusion, face tracking error, light source changes, and the like. However, the above prior art only tries to solve the problem of rigid and non-rigid motion.

A second problem in HR estimation is accuracy deterioration due to failure of face (or body part) tracking. The reason for the occurrence of this problem is, prior art uses face tracker to find location of face in every frame, but on many occasions, this face tracker detects the face location inaccurately. The prior art has no way to identify such tracking failure and noisy data is used for HR estimation leading to low accuracy error.

A third problem in HR estimation is accuracy deterioration in the presence of noise from unknown sources. The reason for the occurrence of this problem is, prior art only removes noise coming from rigid and non-rigid motion performed by person under observation, however, it has no way to identify when noise is coming from other unknown sources. This means that prior art is unable to differentiate noisy pulse from clean pulse signal in the absence of head motion and facial expression. This results in deterioration of HR estimation accuracy in the presence of unknown noise sources.

A fourth problem in HR estimation is accuracy deterioration in the presence of severe head motions or facial expressions. The reason for the occurrence of this problem is, when large head motion and/or facial expression changes occur, the noise is very dominant over cardiac fluctuations and noise correction steps in prior art fail to completely remove noise coming from large head motions and facial expressions. This results in deterioration of HR estimation accuracy.

A fifth problem in HR estimation is accuracy deterioration due to incorrect reference estimate selection. The prior art selects HR estimated in absence of head motion and facial expression as “reference HR” value for performing HR estimation over subsequent video frames which contain head motion and facial expression noise. However, if noise is present due to unknown sources, inaccurate HR estimation leads to an incorrect “reference HR” value. This results in inaccurate HR estimation over a long period which uses the incorrect reference.

In addition, the inventions disclosed in the PTL 2 and the PTL 3 have a problem that a reference periodicity value or repeat base unit has to be identified, but there is no way to ensure that this reference value/unit is reliable or correct. In many cases, when an incorrect reference is selected, it can lead to incorrect HR estimation over a long time period which uses the incorrect reference.

One example of an object of the present disclosure is to provide a correct reference HR (or periodicity value or repeat base unit) is selected and HR estimation accuracy is improved in the presence of noise caused by head motion, facial expression, tracking error and/or unknown sources according to which the above-described problems are eliminated.

### First Exemplary Embodiment

<Device Configuration>

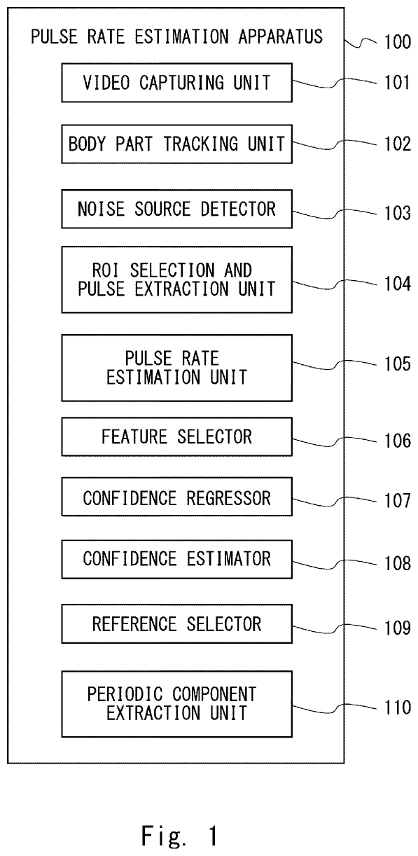

FIG. 1 is a block diagram illustrating a structure of a pulse rate estimation apparatus 100 according to a first exemplary embodiment of the present disclosure. The pulse rate estimation apparatus 100 includes a video capturing unit 101, a body part tracking unit 102, a noise source detector 103, a ROI selection and pulse extraction unit 104, a pulse rate estimation unit 105, a feature selector 106, a confidence regressor 107, a confidence estimator 108, a reference selector 109 and a periodic component extraction unit 110.

The pulse rate estimation apparatus 100 is performed in a training stage and a testing stage. In the training stage, the pulse rate estimation apparatus 100 trains a determination model using a first video data of a first body part for training and a measurement of a physiological information measured from the first body by the video capturing unit 101, the body part tracking unit 102, the noise source detector 103, the ROI selection and pulse extraction unit 104, the pulse rate estimation unit 105, the feature selector 106 and the confidence regressor 107. In the testing stage, the pulse rate estimation apparatus 100 estimates a pulse rate of a second body for estimation using a second video data of the second body part and the trained determination model by the video capturing unit 101, the body part tracking unit 102, the noise source detector 103, the ROI selection and pulse extraction unit 104, the pulse rate estimation unit 105, the feature selector 106, the confidence estimator 108, the reference selector 109 and the periodic component extraction unit 110. Note that, the first body and the second body may be the body of the same person. Otherwise the first body and the second body may be different person's bodies.

The video capturing unit 101 captures a video data (the first or second video data) of a human body (the first or second human body) part from where a skin of the human is exposed. For example, the video capturing unit 101 captures the video data of a human face. Note that, the human body part is an area where a pulse signal, which is the direct effect of a certain physiological process, be extracted.

The body part tracking unit 102 detects a specific human body part, and generates feature points indicating important structural landmarks on the body part for each frame of the video data, captured by the video capturing unit 101, where the body part is detected. For example, the body part tracking unit 102, as face tracker, tracks the face region over time, using skin-color detection or facial landmark detection. A position of facial feature points is used to detect presence of head motion/facial expression and assign appropriate label to each video frame.

Note that, in the following explanation, it is assumed that the body part tracking unit 102 detects human face as the human body part. However, the body part tracking unit 102 of the present disclosure may detect many other body parts where the skin is visible, like hand or ear from the video data. Therefore, the body part tracking unit 102 may be a hand tracker, an ear tracker or the like.

The noise source detector 103 detects noise source based on the feature points detected by the body part tracking unit 102. That is, the noise source detector 103 identifies the noise source for each frame of the video data and assigns labels to each frame.

The ROI selection and pulse extraction unit 104 selects a region(s) of interest (ROI) on the face from the video data based on the feature points and divides each ROI into several ROI sub-regions. The ROI essentially represents periodic fluctuations happening on the color of the face because of physiological activity (e.g. periodic heart-beating activity), henceforth referred to as pulse fluctuations. Further, the ROI selection and pulse extraction unit 104 extracts a pulse signal from each ROI sub-regions. For example, the ROI selection and pulse extraction unit 104 may extract green channel amplitude fluctuations as the pulse signal for each ROI sub-regions.

Further, the ROI selection and pulse extraction unit 104 creates a label-dependent ROI filter to assign each ROI sub-region with a weight proportional to the amount of useful pulse information present in the extracted pulse signal using the assigned labels. The ROI filter is used to suppress sub-regions with high local (temporal) variance/maximum of the extracted pulse signal depending on the assigned labels.

After that, the ROI selection and pulse extraction unit 104 applies the created ROI filter to the extracted pulse signals and performs a label-dependent noise correction to the extracted pulse signals. For example, the ROI selection and pulse extraction unit 104 performs the label-dependent noise correction to remove the noise and to obtain a combined, noise-free pulse signal. Note that, the ROI selection and pulse extraction unit 104 may combines the extracted pulse signals and the created ROI filter.

The pulse rate estimation unit 105 estimates an initial pulse rate frequency by performing frequency analysis on the filtered (extracted) pulse signal. For example, the pulse rate estimation unit 105 may combine the extracted pulse signals and the created ROI filter to form a noise suppressed pulse signal, performs frequency analysis on that extracted noise suppressed pulse signal and generates the estimated initial pulse rate. For example, The pulse rate estimation unit 105 may estimate the initial pulse rate by performing spectral peak tracking to select the correct pulse rate frequency from the set of noisy pulse rate estimate candidates and outputting the initial pulse rate estimates for each video frame.

Moreover, the pulse rate estimation unit 105 outputs feature data to the feature selector 106. Note that, the feature data includes at least one of the estimated initial pulse rate, the detected feature points, the extracted pulse signal, the identified noise source labels, coefficients of the generated ROI filters and results of the frequency analysis.

The feature selector 106 generates high level features from the input data containing the feature data output by the pulse rate estimation unit 105. Note that, the feature data have lower quality than the high level features generated by the feature selector 106. Because the feature data include noisy/corrupted pulse signal. The feature selector 106 is an example of a feature data processing unit. The feature selector 106 performs a predetermined statistic process to reduce noise for the first feature data and output a third feature data. So The feature selector 106 selects the third feature data which has a higher quality among the first feature data, extracts the third feature data from the first feature data, or generates the third feature data based on the first feature data. Note that, the feature selector 106 may perform at least one of color space transforms, a combination of filters and signal decomposition on the first feature data as the predetermined statistic process. For example, the feature selector 106 takes as input the feature data and applies color space transforms, a combination of filters and/or signal decomposition on the input to get the features that are used for confidence level determination. The high level features generated by the feature selector 106 can be used potentially to characterize high accuracy yielding pulse signal, to distinguish it from noisy/corrupted pulse signal and to feed it to the confidence regressor 107 to get a reliability index/confidence value.

The confidence regressor 107 trains a regression analysis model (the determination model) using, as input, the third feature data generated by the feature selector 106 and using, as label, difference between the estimated initial pulse rate (or HR) and a ground truth pulse rate (or HR, the measurement of the physiological information measured from the first human body). The regression analysis model is a computer program module in which a mathematical model is implemented. The regression analysis model inputs the features generated by the feature selector 106, determines a confidence value between 0 and 1 (confidence level/reliability index) which indicates an reliability in the estimation of the initial pulse rate and outputs the confidence value. The confidence value close to 0 means high difference between the estimated initial pulse rate and the ground truth pulse rate of HR, and the confidence value close to 1 means low difference between the estimated initial pulse rate and the ground truth pulse rate of HR.

The confidence regressor 107 trains the regression analysis model so as to minimize difference between the confidence level determined by the determination model and the true confidence level (label, the difference between the estimated initial pulse rate and the ground truth pulse rate). In other words, the confidence regressor 107 trains the regression analysis model to approximate a model function for generating the reliability index (or the confidence level/value), a number between 0 and 1, for each set of video frames under observation, taking as input the features generated by the feature selector 106. Further, the CONFIDENCE REGRESSOR 107 may train the determination model so that the confidence value is determined to be higher as the first pulse rate is closer to the measurement value.

Note that, these reliability indices for training data may be generated manually or automatically, although automatic generation is preferred. For automatic generation of reliability index labels, the confidence regressor 107 may generate the difference between the ground truth pulse rate of HR value and the estimated initial pulse rate value. In that case, the confidence regressor 107 let the regression analysis model learn so that the reliability index become higher as the difference is smaller. Upon completion of the training stage, the trained model can be used in the testing stage for generating reliability indices for the unseen testing data.

When the video capturing unit 101 captures a second video data of the body part for estimation in the testing stage, the body part tracking unit 102, the noise source detector 103, the ROI selection and pulse extraction unit 104 and the pulse rate estimation unit 105 estimate a second initial pulse rate from a second video data. Further, the feature selector 106 generates second high level features from second feature data which includes the second initial pulse rate and other features derived in the estimating the second initial pulse rate.

The confidence estimator 108 acquires the second high level features generated by the feature selector 106 and acquires the confidence value of the second pulse rate using the second high level features and the determination model trained by the confidence regressor 107. That is, the confidence estimator 108 inputs the second high level features (or the second feature data) to the the trained determination model. Then the trained determination model generates a reliability index (or confidence level/value), a number between 0 and 1, for each set of video frames under observation and outputs the generated reliability index to the confidence estimator 108.

The reference selector 109 selects a reference (for example, a reference HR frequency, periodicity value or repeat base unit) for each set of video frames under observation using the reliability indices of past video frames (2-10 seconds), generated by the trained determination model. That is, the reference selector 109 uses the reliability indices of video frames from previous and/or future 2-10 seconds to generate the reference that coarsely characterizes the desired noise-free cardiac fluctuations. The reference is one example of reference periodicity information which is a reference of a period for each frame in the second video data. So the reference selector 109 may select the reference periodicity information based on the acquired confidence value. The reference may be a pulse rate value, a periodicity value or a signal representing a cleaned pulse signal that is expected in the noisy scenario. In the following explanation, it is assumed that the reference selector 109 selects a reference periodicity value as the reference.

The periodic component extraction unit 110 extracts a periodic component out of the pulse signal (sub-regional pulse signals) extracted by the ROI selection and pulse extraction unit 104 using the reference selected by the reference selector 109. Note that, the set of sub-regional pulse signals form a multi-component input signal for the periodic component extraction unit 110. And the periodic component extraction unit 110 performs periodic component extraction or correlation maximization to extract the most periodic component from the input data which is consistent with the reference signal. In other words, the periodic component extraction unit 110 applies adaptive filter as well as uses signal decomposition methods or autocorrelation maximization to extract periodic components from multi-component pulse signal (components being the three color channels (R, G, B), channels other color subspaces (like HSV), or spatial channels, sub-ROIs, on the face) with the aim of extracting noise free pulse signal. The adaptive filtering applies frequency domain or time domain filtering to pulse signal based on confidence level value. And the reference periodicity from the reference selector 109 can be used to determine the cut-off values or bandwidth or other similar parameters of the adaptive filter. Further, the periodic component extraction unit 110 finally gives as output, a final pulse rate (third pulse rate), which is usually the frequency value corresponding to the largest peak in the FFT (Fast Fourier Transform) of the periodic component. That is, the periodic component extraction unit 110 estimates the final pulse rate by performing a frequency analysis on the extracted periodic component. Further, the periodic component extraction unit 110 estimates the third pulse rate using the reference periodicity information. Moreover, the periodic component extraction unit 110 may estimate the third pulse rate by extracting, using the reference periodicity information, at least one of certain periodic components from multicomponent pulse signal which is extracted by the first estimation unit from each frame in the second video data.

<Operations of Apparatus>

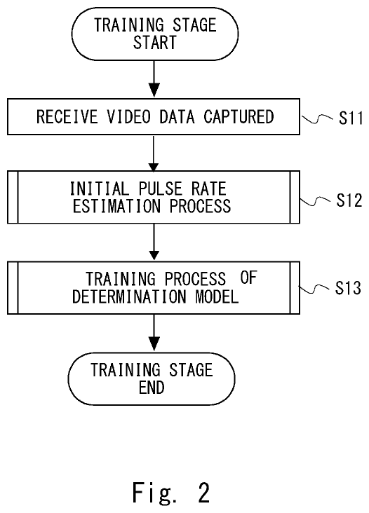

FIG. 2 is a flowchart for explaining the training stage for pulse rate estimation according to the first exemplary embodiment of the present disclosure. In the following explanation, it is assumed that the pulse rate estimation apparatus 100 estimate heart rate from a video of a person's face. Also, in the first exemplary embodiment, the pulse rate estimation method is carried out by allowing the pulse rate estimation apparatus 100 to operate. Accordingly, the description of the pulse rate estimation method of the present embodiment will be substituted with the following description of operations performed by the pulse rate estimation apparatus 100.

First, the video capturing unit 101 captures a first video data of a human face for training. Then the body part tracking unit 102 receives the first video data from the video capturing unit 101 (S11).

Next, the pulse rate estimation apparatus 100 performs an initial pulse rate estimation (S12) using the body part tracking unit 102, the noise source detector 103, ROI selection and pulse extraction unit 104 and the pulse rate estimation unit 105. After that, the pulse rate estimation apparatus 100 performs a determination model training (S13) using the feature selector 106 and the confidence regressor 107.

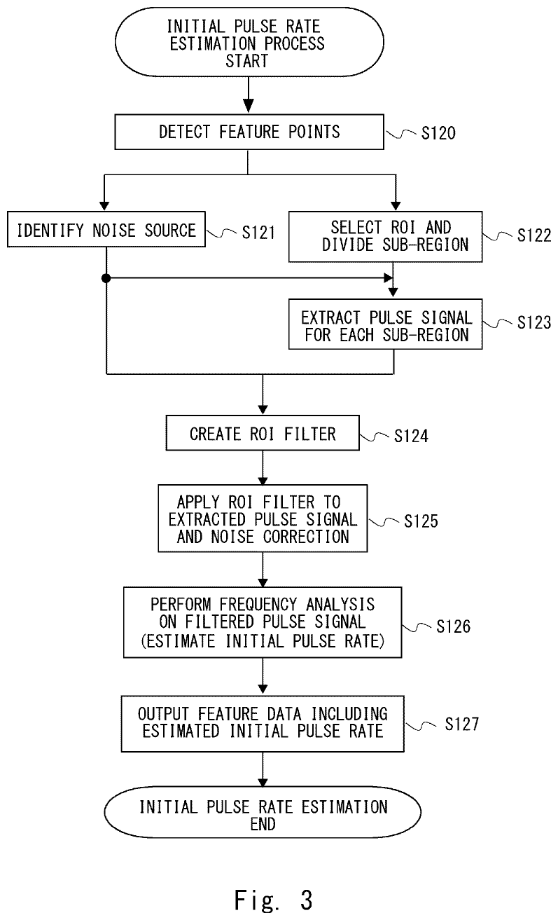

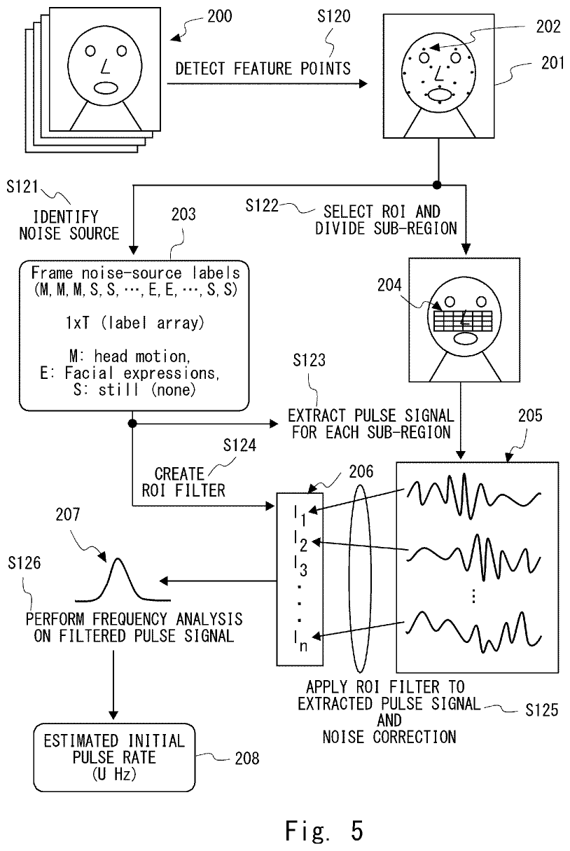

FIG. 3 is a flowchart for explaining the initial pulse rate estimation processing according to the first exemplary embodiment of the present disclosure and FIG. 5 is a diagram for explaining a concept of a process to estimate the initial pulse rate from captured images.

First, the body part tracking unit 102 detects feature points from each frames of captured images 200 (the first video data) (S120). That is, the body part tracking unit 102 tracks the face of the first person being observed (for training) for each video frame. For example, the body part tracking unit 102 detects feature points 202 in the captured image 201 which is one of the captured images 200.

Next, the noise source detector 103 identifies noise source in each frame based on the feature points (S121) and assigns frame noise source labels 203 to each frame. That is, the noise source detector 103 identifies noise source in the captured image 201 using the feature points 202 and assigns one of labels which is a kind of noise sources to a frame of the captured image 201. For example, the frame noise source labels may include three labels M, E and S. The label M indicates that the noise source of the frame is a “head motion”. The label E indicates that the noise source of the frame is “facial expressions”. The label S indicates that noise sources were not “still” identified from the frame, that is the noise source of the frame is “none”. After that the noise source detector 103 outputs the assigned labels to the ROI selection and pulse extraction unit 104.

Simultaneously, the ROI selection and pulse extraction unit 104 selects ROIs on the face and divides each ROI into several ROI sub-regions (S122) for the localization of noise and pulse information. For example, the ROI selection and pulse extraction unit 104 selects a ROI on the face from the captured image 201 based on the feature points 202 and divides the ROI into several ROI sub regions 204.

After the steps S121 and S122, the ROI selection and pulse extraction unit 104 extracts a pulse signal from each ROI sub-regions (S123). For example, the ROI selection and pulse extraction unit 104 extracts pulse signals 205 from the ROI sub regions 204 using the noise source labels 203.

After the steps S121 and S123, the ROI selection and pulse extraction unit 104 creates a ROI filter to assign each ROI sub-region with a weight proportional to the amount of useful pulse information present in the extracted pulse signal using the assigned labels (S124). For example, the ROI selection and pulse extraction unit 104 creates a ROI filter 206 using the noise source labels 203 and the extracted pulse signal 205. The ROI filter 206 includes several coefficients l₁, l₂, l₃, . . . , lₙ (n is a natural number of 2 or more). For example, the coefficient l₁ corresponds to a first pulse signal in the extracted pulse signal 205, the first pulse signal is extracted, at the step S123, from the ROI sub-region of a first frame (captured image) which is included in the captured images 200. And the coefficient l₁ is derived based on the noise source label which corresponds to the first frame. The coefficient l₂ corresponds to a second pulse signal in the extracted pulse signal 205, and it is the same after that.

After the step S124, the ROI selection and pulse extraction unit 104 applies the created ROI filter to the extracted pulse signals and performs a label-dependent noise correction to the extracted pulse signals (S125). For example, the ROI selection and pulse extraction unit 104 applies the ROI filter 206 to the extracted pulse signal 205, performs label-specific correction to remove the noise and to obtain a noise-free pulse signal and outputs a noise suppressed pulse signal as a filtered pulse signal 207.

After the step S125, the pulse rate estimation unit 105 performs frequency analysis on that extracted noise suppressed pulse signal (combined pulse signal) and generating a pulse rate estimate also known as the “simple estimate”. That is, the pulse rate estimation unit 105 estimates an initial estimated pulse rate 208 (S126) by performing frequency analysis on the filtered pulse signal 207. For example, the initial estimated pulse rate may be a frequency value corresponding to a highest peak of the FFT of the combined pulse signal.

After the step S126, the pulse rate estimation unit 105 outputs (first) feature data (S127), which includes at least one of the estimated initial pulse rate, the detected feature points (feature point locations), the extracted pulse signal, the identified noise source labels, coefficients of the generated ROI filters and results of the frequency analysis (FFT), to the feature selector 106.

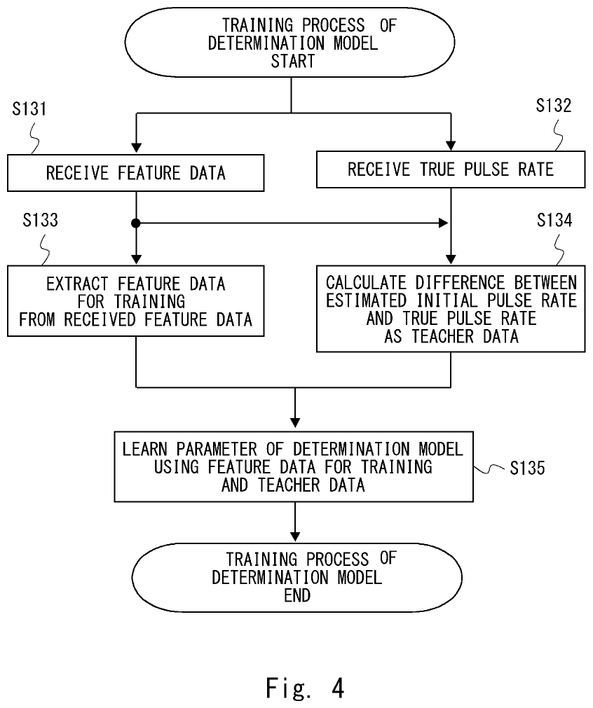

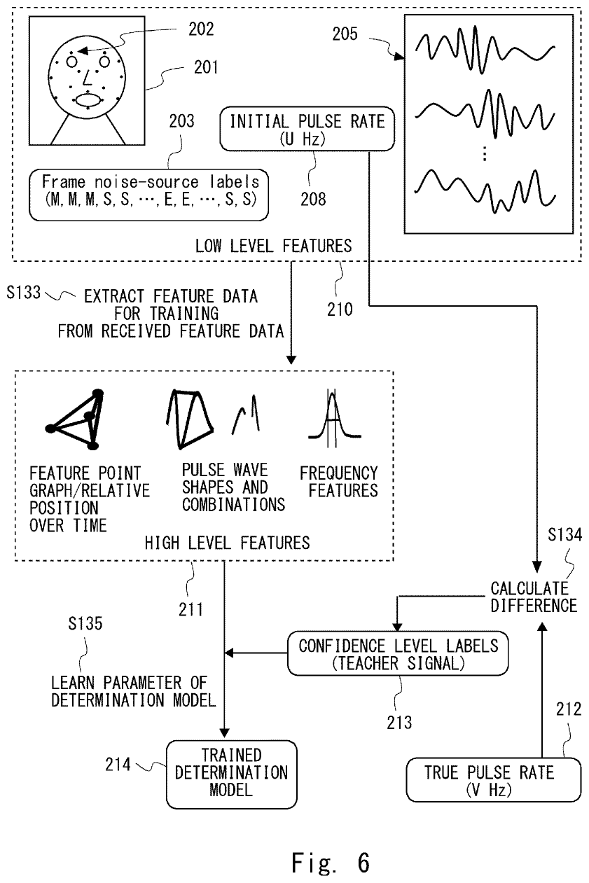

FIG. 4 is a flowchart for explaining the training process of determination model according to the first exemplary embodiment of the present disclosure and FIG. 6 is a diagram for explaining a concept of a process to train the determination model from feature data.

First, the feature selector 106 receives the first feature data output from the pulse rate estimation unit 105 (S131). Note that, because the first feature data include noisy/corrupted pulse signal, it can be said low level features than output data by the feature selector 106. So the low level features 210 include the feature points 202, the noise source labels 203, the extracted pulse signal 205, the initial estimated pulse rate 208 and the like.

Next, the feature selector 106 extracts (third) feature data for training from the received first feature data (S133). Note that, the third feature data have higher quality than the first feature data. That is, the first feature data is low-level features 210 and the third feature data is high-level features 211. The third feature data can be used potentially to characterize high accuracy yielding pulse signal, to distinguish it from noisy/corrupted pulse signal and to feed it to the confidence regressor 107 to get a reliability index/confidence value. For example, the high level features 211 may include feature point graph/relative position overtime, pulse wave shapes and combinations, frequency features or the like. In other words, the feature selector 106 extracts high level features explicitly or implicitly (with the Neural network regressor), to get more useful information (features that capture pulse wave shape, or noise characteristics of pulse wave or feature point locations, etc.). So the third feature data may include a part of the feature points 202, the noise source labels 203, the extracted pulse signal 205, the initial estimated pulse rate 208 and the like.

Not depending on the step S131, the confidence regressor 107 receives the true pulse rate (S132). The true pulse rate 212 is an example of the physiological information measured from the first body and a measurement value which is a pulse rate measured from the body during capturing the first video data, for example, using a gold standard pulse rate measurement device.

After the steps S131 and S132, the confidence regressor 107 calculates a difference between the estimated initial pulse rate and the true pulse rate as a teacher data (S134). For example, the confidence regressor 107 computes the difference between the initial estimated pulse rate 208 (U Hz) and the true pulse rate 212 (V Hz) as a confidence level labels 213.

After the steps S133 and S134, the confidence regressor 107 learns parameters of the regression analysis model using the third feature data (high level features 211) for training and the teacher data (confidence level labels 213) (S135). For example, the confidence regressor 107 learns a distribution of input features by a network/model. The regression model can generate a reliability index (a value between 0 and 1) for the extracted features, which is a measure of how much a set of features can be considered non-noisy and depends on how much the simple estimate agrees with the true pulse rate. The confidence regressor 107 trains the regression model so as to minimize the difference between true confidence level (the teacher data) and the determined confidence level, by penalizing inaccurate estimations and rewarding accurate ones. In other words, the confidence regressor 107 learns the distribution of high-level features to get a scalar value (confidence level) between 0 and 1, corresponding to how reliable the input video stream is at a certain time. For example, the confidence regressor 107 learns weights (w) of the confidence level regression analysis model to output correct confidence level, for example, using a following expression:

Minimize∥w*(input feature vector)−Y∥\hat{ }2.

Note that, the confidence regressor 107 may output the trained regression model (trained determination model 214) to a storage device (not shown) in the pulse rate estimation apparatus 100.

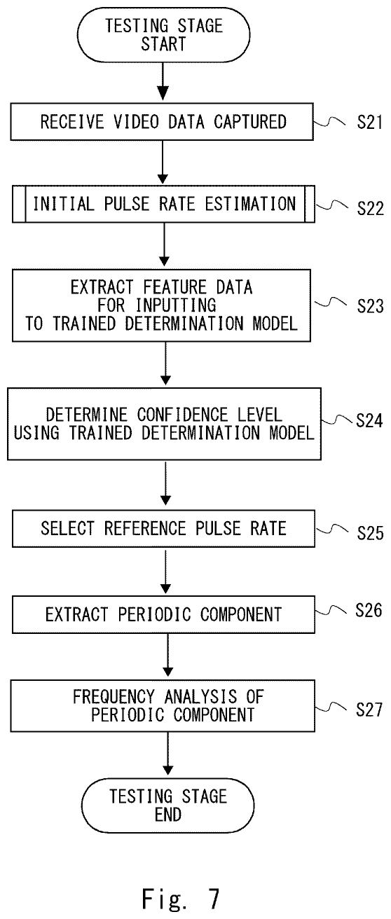

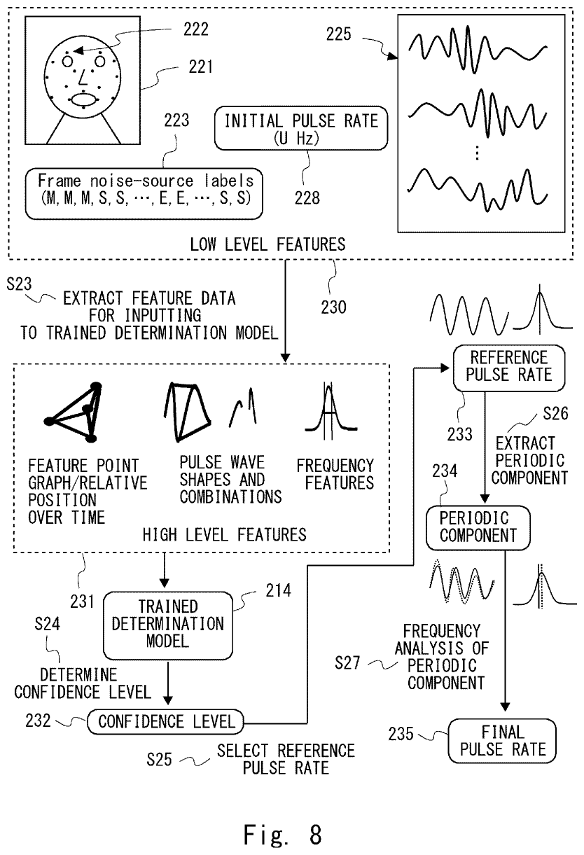

FIG. 7 is a flowchart for explaining the testing stage for pulse rate estimation according to the first exemplary embodiment of the present disclosure and FIG. 8 is a diagram for explaining a concept of a process to estimate a final pulse rate from an initial pulse rate at the testing state.

First, the body part tracking unit 102 detects feature points from each frames of captured images (the second video data) (S21). Note that, the second video data are captured images whose pulse signals are unseen and are data for estimation of the pulse signal. That is, the body part tracking unit 102 tracks the face of the second person being observed (for estimation) for each video frame. For example, the body part tracking unit 102 detects feature points 222 in the captured image 221 which is one of the captured images for estimation.

Next, as in the steps S120 to S127 in FIG. 3, the pulse rate estimation apparatus 100 performs an initial pulse rate estimation (S22) using the body part tracking unit 102, the noise source detector 103, ROI selection and pulse extraction unit 104 and the pulse rate estimation unit 105. As a result, the pulse rate estimation unit 105 estimates the second initial pulse rate from a second video data and outputs the second feature data, which includes the second initial pulse rate and other features derived in the estimating the second initial pulse rate, to the feature selector 106. For example, the second feature data are a low level features 230 which include the feature points 222, the noise source labels 223, the extracted pulse signal 225, the initial estimated pulse rate 228 and the like.

After the step S22, as in the steps S131 and S133 in FIG. 4, the feature selector 106 receives the second feature data and extracts (fourth) feature data for inputting to the trained determination model 214 from the received second feature data (S23). The fourth feature data may include a part of the feature points 222, the noise source labels 223, the extracted pulse signal 225, the initial estimated pulse rate 228 and the like.

After the step S23, the confidence estimator 108 determines a confidence level of the second pulse rate using the trained determination model 214 (S24). The trained determination model 214, that is the trained regressor or the trained regression analysis model, had been set(/fixed) the weights, which are the parameters learned by the confidence regressor 107 at the step S135 in FIG. 4. That is, the trained determination model 214 had already learnt weights (w) of the regression analysis model to output correct confidence level.

More specifically, the confidence estimator 108 acquires the second high level features 211 (the fourth feature data) from the feature selector 106, inputs the second high level features 211 to the trained determination model 214. The trained determination model 214 generates a confidence level 232 from the second high level features 211, for example, using a following expression:

Confidence level=w*(input feature vector).

And the trained determination model 214 outputs the confidence level 232 to the confidence estimator 108. So, the confidence estimator 108 acquires the confidence level 232. For example, the confidence level 232 is a scalar value (between 0 and 1) representing the confidence value of the second video data. The confidence level is used by the reference selector 109 and the periodic component extraction unit 110 for extracting the cardiac fluctuations from the pulse signal in the presence of noise.

After the step S24, the reference selector 109 selects a reference pulse rate 233 for each set of frames(second video data) using the confidence level 232 (S25). For example, the reference selector 109 selects a representative frequency value around which a finer frequency analysis can be performed to get final pulse rate. The the confidence level 232 of neighbor frames (from past and/or future) are used to select the reference (Coarse-level estimation). In other words, the reference selector 109 selects the reference pulse signal or pulse rate (frequency/periodicity) value. That is, the reference selection means that the final estimated pulse (rate) is expected to be similar to the reference as the reference is selected from high confidence input features in the near past and/or future. In other words, the reference selector 109 generates the reference that coarsely characterizes the desired noise-free cardiac fluctuations by using the reliability indices of the second video frames from previous and/or future 2-10 seconds. The reference can be a pulse rate value or a periodicity value or a signal representing a cleaned pulse signal that is expected in the noisy scenario. Note that, in the present embodiments, it is assumed that the reference pulse rate value is selected.

After the step S25, the periodic component extraction unit 110 extracts a periodic component 234 (S26) out of the pulse signal (sub-regional pulse signals) extracted by the ROI selection and pulse extraction unit 104 using the reference selected by the reference selector 109. That is, the periodic component extraction unit 110 extracts the most periodic components of the pulse wave using selected reference frequency. In other words, the periodic component extraction unit 110 performs a fine-level estimation using the reference frequency, a finer frequency analysis is performed to get final pulse rate. For example, the periodic component extraction unit 110 extracts the most periodic component from the noisy input features which is close to the selected reference (a rate near the selected reference rate or a pulse wave which closely resembles the selected reference signal). Another example, the periodic component extraction unit 110 uses adaptive filtering, signal decomposition methods and/or autocorrelation maximization with the help of the coarse reference generated by the reference selector 109 to extract periodic components from multi-component pulse signal (components being the three color channels (R, G, B), channels other color subspaces (like HSV, YCbCr, etc.), or spatial channels, sub-ROIs, on the face) with the aim of extracting noise free pulse signal.

After the step S25, the periodic component extraction unit 110 performs a frequency analysis of the periodic component 234 and outputs a final pulse rate 235 (S27). That is, the periodic component extraction unit 110 estimates the final pulse rate 235 (third pulse rate) by performing the frequency analysis of the periodic component 234.

<Pulse Rate Estimation from a Video Sequence>

The first estimation unit includes (the video capturing unit 101) the body part tracking unit 102, the noise source detector 103, the ROI selection and pulse extraction unit 104 and the pulse rate estimation unit 105. The first estimation unit performs “simple pulse rate estimation” on any sequence of images, taking as input, a pre-recorded video (sequence of images) or a live video recording stream from a web camera or an infrared camera or any video capturing device. Further the first estimation unit generates an output which is the estimated pulse signal. In this process, the first estimation unit generates many features describing the pulse signal such as the noise source, the ROI filter weights, the pulse signal statistics (mean, variance, etc.), the FFT of the pulse signal, the SNR of the pulse signal and so on. The estimated pulse signal, along with the features generated in the process of the simple pulse rate estimation, are used as low level features for generating high level features in the feature selector 106. We explain the working of the feature selector 106 below.

<Feature Selection>

The low level features generated during the signal processing in the first estimation unit contain information about to what extent the pulse signal is corrupted by noise. However, not all of these features are useful or relevant to determine the effect of noise (or the reliability index). So, we need to identify features (or create high-level features) which will give us more information about the noise and select only those features as output of the feature selector 106 to make the regression analysis more accurate.

There are multiple ways to select relevant features:

i) manually select/create the features which contain noise-related information like SNR (ratio of power spectral density (PSD) around largest peak and PSD over the rest of the pulse rate frequency band), mean and variance of pulse signal, etc.;

ii) perform explicit feature selection/elimination using filter and wrapper methods like PCA/ICA/LDA, LASSO regression, CFS, etc.;

iii) an implicit feature selection performed by a regression analysis technique or neural network used for regression in the training stage.

The first embodiment of the present disclosure performs implicit feature selection (method (iii) above) via a neural network that is trained to assign confidence level to the input features by performing regression analysis in the training stage.

<Training Stage—Training Regression Model for Confidence Level>

The training stage is a period where the confidence regressor 107 trains the regression analysis model(/network) to learn the distribution of input features and assign them with a confidence level/reliability index (value between 0 and 1). The true labels (true value of confidence) are generated with the knowledge of the true pulse rate which can be measured using a gold standard pulse rate measurement device (for instance, a gold standard ECG device in case of heart rate). The true confidence level value close to 0 means high difference between the estimated initial pulse rate and the ground truth value of HR, and the true confidence level value close to 1 means low difference between the estimated initial pulse rate and the ground truth value of HR. The estimated initial pulse rate is the pulse rate estimated by the pulse rate estimation unit 105 using the above “simple pulse rate estimation” methods.

The first embodiment of the present disclosure uses the formula given in equation (1) for calculating the true confidence level (which acts as the “teacher signal” for training the regression analysis model) for input features obtained at time t. However, any measure that indicates how close (or how far) the estimated initial pulse rate is to the ground truth pulse rate value measured using the gold standard pulse rate measurement device can be used as the true confidence level.

$$\begin{matrix} [ Math . 1 ] & \\ Confidence Level ( t ) = min ( 1 , \frac{2}{\vert True Rate ( t ) - Estimated Rate ( t ) \vert} ) & ( 1 )\end{matrix}$$

The regression analysis model is trained to minimize difference between the true confidence level and the determined confidence level, by penalizing inaccurate estimations and rewarding accurate ones.

In this process, the weights of the regression analysis model (for example, the weight coefficients in a linear regression model) are optimized in order to minimize the error between confidence level predicted by the model and the true confidence level. Equations (2) and (3) show how this is done for least-squares regression with input data matrix X (n×m, n data-points, m features), true label column matrix Y (n×1) and weight vector W (m×1). We need to find optimal weights W* such that the error between XW and Y is minimized.

[Math. 2]

W*==arg min(∥Y−XW∥²) (2)

[Math. 3]

W*=(X^{T}X)⁻¹X^{T} (3)

After the training is complete, the confidence regressor 107 freezes the weights W* of the regression model and use it in the testing stage as the confidence estimator 108. Any new input

[symbol]

(1×m) [Math. 4]

will be assigned a confidence level

Ŷₜ=W^{T} [symbol] [Math. 5]

in the case of a trained least-squares regressor. Along the same lines of regression analysis, in the first embodiment of the present disclosure, the confidence regressor 107 may use a more sophisticated RNN (Recurrent Neural Network)-based regressor for learning the confidence level distribution over input features.

<Testing Stage>

The testing stage comes after the training stage. The testing stage consists of the same initial steps as the training stage by using the video capturing unit 101, the body part tracking unit 102, the noise source detector 103, the ROI selection and pulse extraction unit 104, the pulse rate estimation unit 105 and the feature selector 106. Since after the completion of training stage, the determination model has been trained, the confidence estimator 108 uses it to continuously estimate and update the confidence level of new (unseen) input features, estimating confidence level values for windows of short time length (2 seconds-4 seconds) as well as long time lengths (30 seconds-60 seconds) as per need. Followed by this, the reference selector 109 performs reference selection to select a reference (periodicity value, pulse rate value or repeat base unit, a.k.a. cyclet) value that coarsely represents the desired cardiac fluctuations using the confidence values estimated in the confidence estimator 108 and finally the periodic component extraction unit 110 performs periodic component extraction to get the component that best represents the desired cardiac fluctuations. The frequency (i.e. rate=1/periodicity) of this periodic component is used to get the final pulse rate estimate. Each component used in the testing stage after the feature selector 106 is explained in greater detail below.

<Confidence Level Estimation>

Once the determination model has been trained and the weights W* locked, it is used in the testing phase by the confidence estimator 108. For the simple case of least-squares regressor represented by equations (2) and (3), the confidence level estimate

[symbol]

[Math. 6]

(scalar between 0 and 1) of any new, unseen input feature set

[symbol]

(1×m), [Math. 7]

representing a small length of video input, will be given by

[symbol]

=W^{T} [symbol] . [Math. 8]

A trained RNN-based regressor will also perform in a similar manner, taking in as input a feature set generated in the feature selector 106 and giving out as output a scalar value per input feature set. This confidence level will represent the reliability of the input features (and thus the reliability of the pulse rate estimate generated by the simple pulse rate estimation technique (the first estimation unit) of a small part of a long video sequence, for instance, the video clip between time t₁ and time t₁+4 seconds, in the long video sequence. This way, at time t₁ in a long video sequence from the test data, the confidence estimator 108 will have estimated confidence levels over all times up to time t₁, i.e. at all times from time 0 seconds to time t₁. The reference selector 109 uses these confidence levels from the past (if the estimation is being performed in real-time) as well as from the future (if the estimation is not real-time, being performed at a later time) to select a reliable reference (periodicity value, pulse rate value or repeat base unit, a.k.a. cyclet) to coarsely represent the cardiac fluctuations present in the obtained pulse signal (by the ROI selection and pulse extraction unit 104) at time t₁ in the video.

<Reference Selection>

The reference selection is the procedure of selecting the defining characteristic of the signal that would be used for coarse level estimation of the desired pulse signal. For example, if we are confident that the pulse rate is between 0.2 to 0.3 Hz, a reference will be the rate 0.25 Hz and a narrow band-pass filter centered at 0.25 Hz with bandwidth of 0.1 Hz can be used to find the cardiac fluctuations (in case of HR estimation) in a noisy pulse signal. The reference helps us localize the pulse signal (in frequency) and discard the false pulse rate values (those which are far away from the reference pulse rate value) which could be mistaken as the pulse rate in the absence of a reference. Since the final estimated pulse rate depends heavily on the reference, it is necessary that the reference is selected accurately. Prior methods of the PTL 2 and PTL 3 calculate a reference as the mean pulse rate over a short/long time or as a repeat base unit (a cyclet) which has the highest autocorrelation value over a short/long time, but the reference calculated that way is susceptible to being inaccurate, especially in the presence of noise (if reference calculation is done using short time analysis) or in the presence of fast changing pulse rate (if reference calculation is done using long time analysis).

Hence, the present embodiment uses the confidence levels estimated in the past (if real time processing) and future (if processing is done at a later time) time instances in the video to get calculate the reference (a reference pulse rate value in the first embodiment of the new invention) at each time t₁ as per equation (4).

$$\begin{matrix} [ Math . 9 ] & \\ Reference Rate ( {t}_{1} ) = {Σ}_{t = {t}_{1} - p}^{{t}_{1} + f} \frac{1}{\vert {t}_{1} - t \vert} * \frac{Confidence Level ( t )}{Normalizing Factor} * Estimated Rate ( t ) & ( 4 )\end{matrix}$$

This reference rate is calculated with the following principles and any other reference that follows these principles can be used instead:

i. Reference is calculated using the estimated pulse rate values (obtained from the pulse rate estimation unit 105) which are within the time range [t₁−p, t₁+f], i.e. based on the estimated initial pulse rate (or other features) in a usually small time window around t₁, ranging from p seconds in the past to f seconds in the future. f=0 if pulse rate is being estimated in real time (without lag).

ii. More importance is given to estimated rates at time t close to time t₁

iii. More importance is given to estimates with high confidence levels

The reference rate calculated in this way takes into account, the reliability of the estimates as well as their time-distance from the current time t₁. Hence, this reference rate gives us a coarse idea of the estimated pulse rate at time t₁ with the help of estimated rates and reliability labels of these estimates lying in a small time window around t₁. Next the periodic component extraction unit 110 performs a finer analysis around the reference to more accurately estimate the pulse rate at time t₁.

<Periodic Component Extraction>

Using the reference (pulse rate) obtained by the reference selector 109, the periodic component extraction unit 110 uses narrow band frequency analysis (for example, a narrow band band-pass filter) around the reference pulse rate or a correlation maximization or a periodic component analysis to extract the most periodic component in a small frequency range around the reference frequency. In other words, these periodic component extraction techniques mentioned above make use of the reference in order to find a periodic component (since pulse is periodic/quasi-periodic) that is most prominent/powerful in the multi-component pulse signal obtained by the ROI selection and pulse extraction unit 104. These components can be obtained from the color channels (R, G, B), channels of other color-spaces (like HSV), or spatial channels, also known as sub-ROIs on the face. A narrow band band-pass filter, for instance, will remove the possibility of any estimate that lies outside this narrow band being selected as the final estimate. So, noisy, inaccurate estimates (generated due to any source) will be discarded if they don't agree with the reference pulse rate, leading to an increase in the pulse rate estimation accuracy.

<Final Pulse Rate Estimation>

The final pulse rate 235 is obtained by performing frequency analysis on the extracted periodic component, output of the periodic component extraction unit 110. Generally the highest peak of the FFT of this periodic component (which is the reciprocal of its periodicity) is chosen as the final pulse rate estimate.

### Effects of the Present Embodiment

A first effect is to ensure that it is possible to estimate HR with high accuracy deterioration in the presence of noise from unknown sources. According to the present embodiment, by training a regression analysis model/network to learn the distribution of features that result in an accurate pulse rate estimate and distinguish those features from those which generate inaccurate pulse rate estimate, it is able to successfully detect and quantize noise being introduce by several uncontrollable noise sources. These noise sources include rigid and non-rigid motion performed by the person under observation, occlusion, face tracking error, light source changes, etc. which introduce complex corruption to observed signal and make it difficult to estimate HR, but by using the trained regression model, the present disclosure is able to accurately estimate reliability of new pulse signal, quantize the extent of corruption introduced by noise as well as select an accurate reference for periodic component extraction in.

A second effect is to ensure that it is possible to estimate HR with high accuracy even in the event of failure of face (or body part) tracking. According to the present embodiment, unlike prior art, by using the regression analysis to learn the distribution of corrupted pulse signals, the present disclosure has a way to identify such tracking failure and remove noise by assigning low confidence to noisy data. High confidence data dominates the pulse rate estimation process, hence corrupted data due to face tracking failure, which goes undetected in prior art, doesn't contribute significantly towards pulse rate estimation, resulting in higher accuracy.

A third effect is to ensure that it is possible to estimate HR with high accuracy even in the presence of noise from unknown sources. According to the present embodiment, unlike prior art, by using the regression analysis to learn the distribution of corrupted pulse signals, the present disclosure has a way to identify and remove noise coming from unknown sources by assigning low confidence to noisy data. High confidence data dominates the pulse rate estimation process, hence corrupted data due to unknown noise sources, which goes undetected in prior art, doesn't contribute significantly towards pulse rate estimation, resulting in higher accuracy.

A fourth effect is to ensure that it is possible to estimate HR with high accuracy even in the presence of severe head motions or facial expressions. According to the present embodiment, unlike prior art, by using the regression analysis to learn the distribution of corrupted pulse signals, the present disclosure has a way to identify and remove which is highly dominant, as noisy data has features which are very distinguishable from noise-free data and hence get very low confidence. High confidence data dominates the pulse rate estimation process, hence data affected by severe head motion and/or facial expression, which dominates pulse rate estimation process prior art, doesn't contribute significantly towards pulse rate estimation, resulting in higher accuracy.

A fifth effect is to ensure that it is possible to estimate HR accurately by reducing the cases of incorrect reference estimate selection. While the prior art only requires the absence of head motion and facial expression for a pulse rate estimate to be considered reliable and treats them as “reference HR”, according to the present embodiment, the reference rate is selected using estimates that have been assigned high confidence (by a trained regression model based on features specifically selected to identify noise-free sources) in the recent past (or future). In the case where noise is present due to unknown sources, prior art is susceptible to selecting inaccurate “reference HR” due to its naive criteria. On the other hand, due to the sophisticated procedure of assigning confidence and selecting reference rates in the present disclosure means that accurate reference rate would be selected even in the presence of noise due to unknown sources. Since final pulse rate estimate is heavily dependent on the reference rate value, more accurate reference selection means a more accurate pulse rate estimation. Alternatively, same processing can be performed by using near infrared cameras or infrared cameras and using output pixel values of these imaging devices. In that case, it is possible to obtain the effect.

As described above, according to the present invention, it is possible to improve HR estimation accuracy in the presence of noise caused by head motion, facial expression, tracking error and/or unknown sources. The main advantage of the new invention over NPL1 and PTL1 is that in addition to relative spatial reliability determination of video data (through dynamic ROI selection—similar to NPL1 and PTL1), the new invention also performs absolute temporal reliability determination (through confidence regressor and confidence estimator) of the video data which helps the new invention to

i. Detect whether pulse signal is corrupted by noise (independent of noise-source)

ii. Quantify the level of corruption caused by noise (using confidence level/reliability index)

iii. Remove this noise (by choosing reliable reference with high confidence and low corruption) and extract the component that best represents noise-free cardiac fluctuations (by using adaptive filters and periodic component extraction).

<Program>

A program of the present embodiment need only be a program for causing a computer to execute the required steps shown in FIGS. 2, 3, 4 and 7. The pulse rate estimation apparatus 100 and the pulse rate estimation method according to the present embodiment can be realized by installing the program on a computer and executing it. In this case, the Processor of the computer functions as the video capturing unit 101, the body part tracking unit 102, the noise source detector 103, the ROI selection and pulse extraction unit 104, the pulse rate estimation unit 105, the feature selector 106, the confidence regressor 107, the confidence estimator 108, the reference selector 109, and the periodic component extraction unit 110.

The program according to the present exemplary embodiment may be executed by a computer system constructed using a plurality of computers. In this case, for example, each computer may function as a different one of the video capturing unit 101, the body part tracking unit 102, the noise source detector 103, the ROI selection and pulse extraction unit 104, the pulse rate estimation unit 105, the feature selector 106, the confidence regressor 107, the confidence estimator 108, the reference selector 109, and the periodic component extraction unit 110.

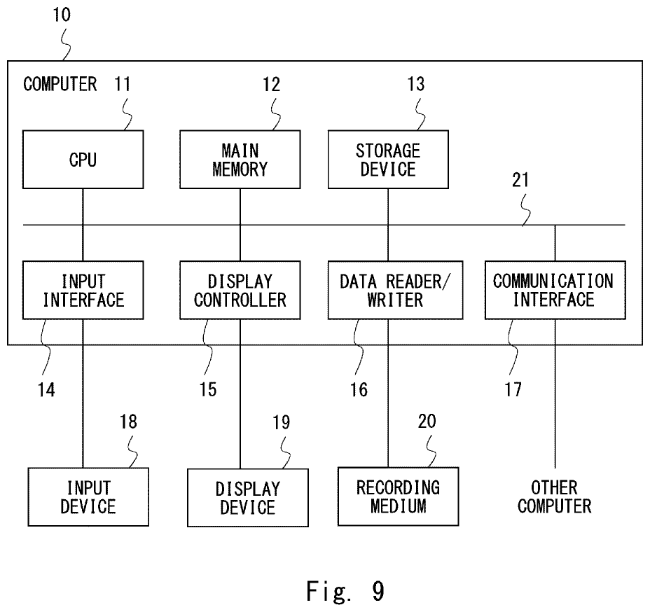

Also, a computer that realizes the pulse rate estimation apparatus 100 by executing the program according to the present embodiment will be described with reference to the drawings. FIG. 9 is a block diagram illustrating a hardware structure of the pulse rate estimation apparatus according to the first exemplary embodiment of the present disclosure.

As shown in FIG. 9, the computer 10 includes a CPU (Central Processing Unit) 11, a main memory 12, a storage device 13, an input interface 14, a display controller 15, a data reader/writer 16, and a communication interface 17. These units are connected via a bus 21 so as to be capable of mutual data communication.

The CPU 11 carries out various calculations by expanding programs (codes) according to the present embodiment, which are stored in the storage device 13, to the main memory 12 and executing them in a predetermined sequence. The main memory 12 is typically a volatile storage device such as a DRAM (Dynamic Random Access Memory). Also, the program according to the present embodiment is provided in a state of being stored in a computer-readable storage medium (recording medium) 20. Note that the program according to the present embodiment may be distributed over the Internet, which is connected to via the communication interface 17.

Also, specific examples of the storage device 13 include a semiconductor storage device such as a flash memory, in addition to a hard disk drive. The input interface 14 mediates data transmission between the CPU 11 and an input device 18 such as a keyboard or a mouse. The display controller 15 is connected to a display device 19 and controls display on the display device 118.

The data reader/writer 16 mediates data transmission between the CPU 11 and the storage medium 20, reads out programs from the storage medium 20, and writes results of processing performed by the computer 10 in the storage medium 20. The communication interface 17 mediates data transmission between the CPU 11 and another computer.

Also, specific examples of the storage medium 20 include a general-purpose semiconductor storage device such as CF (Compact Flash (registered trademark)) and SD (Secure Digital), a magnetic storage medium such as a flexible disk, and an optical storage medium such as a CD-ROM (Compact Disk Read Only Memory).

The pulse rate estimation apparatus 100 according to the present exemplary embodiment can also be realized using items of hardware corresponding to various components, rather than using the computer having the program installed therein. Furthermore, a part of the pulse rate estimation apparatus 100 may be realized by the program, and the remaining part of the pulse rate estimation apparatus 100 may be realized by hardware.

### Second Exemplary Embodiment

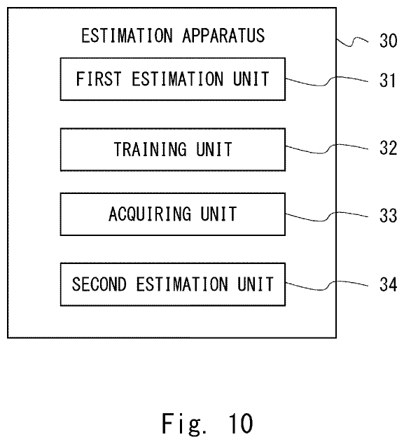

FIG. 10 is a block diagram illustrating a structure of an estimation apparatus 30 according to a second exemplary embodiment of the present disclosure. The estimation apparatus 30 includes a first estimation unit 31, a training unit 32, an acquiring unit 33 and a second estimation unit 34.

The first estimation unit 31 estimates a first pulse rate from a first video data which is captured a body part where a skin is exposed and output a first feature data derived based on the first video data in order to estimate the first pulse rate. Note that, the video capturing unit 101, the body part tracking unit 102, the noise source detector 103, the ROI selection and pulse extraction unit 104 and the pulse rate estimation unit 105 are one example of the first estimation unit 31.

The training unit 32 trains a determination model to determine a confidence value which indicates an reliability in the estimation of the first pulse rate based on the first feature data and a physiological information measured from the body. Note that, the feature selector 106 and the confidence regressor 107 are one example of the training unit 32.

The acquiring unit 33 acquires a second feature data derived by the first estimation unit 31 when the first estimation unit 31 estimates a second pulse rate from a second video data which is captured the body part to be estimated, and acquires the confidence value of the second pulse rate using the second feature data and the determination model trained by the training unit. Note that, the confidence estimator 108 is one example of the acquiring unit 33.

The second estimation unit 34 estimates a third pulse rate based on the acquired confidence value. Note that, the reference selector 109 and the periodic component extraction unit 110 are one example of the second estimation unit 34.

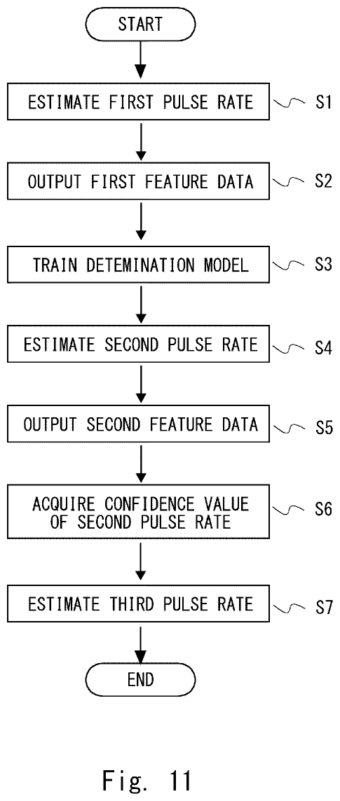

FIG. 11 is a flowchart for explaining an estimation method according to the second exemplary embodiment of the present disclosure. First, the first estimation unit 31 performs a first estimation process to estimate a first pulse rate from a first video data which is captured a body part where a skin is exposed (S1). Next, the first estimation unit 31 outputs a first feature data derived based on the first video data in the first estimation process to the training unit 32 (S2). Further, the training unit 32 trains a determination model to determine a confidence value which indicates a reliability in the estimation of the first pulse rate based on the first feature data and a physiological information measured from the body (S3). Further, the first estimation unit 31 performs a second estimation process to estimate a second pulse rate from a second video data which is captured the body part to be estimated (S4). And the first estimation unit 31 outputs a second feature data derived based on the second video data in the second estimation process to the acquiring unit 33 (S5). After that, the acquiring unit 33 acquires the confidence value of the second pulse rate using the second feature data and the trained determination model (S6). Then the second estimation unit 34 estimates a third pulse rate based on the acquired confidence value (S7).

According to the second exemplary embodiment, it is possible to improve the estimation accuracy.

### Other Exemplary Embodiments of the Invention

Those skilled in the art will recognize that the system, operation and method of the present disclosure may be implemented in several manners and as such are not to be limited by the foregoing embodiments and examples. In other words, functional elements being performed by single or multiple components in various combinations of hardware, software or firmware may be distributed among software applications in the server side (the SP side). Furthermore, the embodiments of the methods presented in the flowchart in this disclosure are provided by way of example in order to provide a more complete understanding of the technology. Alternative embodiments can be contemplated wherein the various components can be altered functionally in order to attain the same goals. Although, various embodiments have been described for the purposes of this disclosure, such embodiments should not be deemed to limit the teaching of this disclosure to those embodiments. Various changes and modifications may be made to the elements and operations described above to obtain a result that remains within the scope of the systems and operations described in this disclosure.

Additionally, it is obvious that the present invention is not limited by the above exemplary embodiments but various modifications can be made thereto without departing from the scope of the already mentioned present invention. For example, the above exemplary embodiments explained the present invention as being a hardware configuration, but the present invention is not limited to this. The present invention can also be realized by causing a CPU (Central Processing Unit) to execute arbitrary processes on a computer program. In this case, the program can be stored and provided to a computer using any type of non-transitory computer readable media.

Examples of non-transitory computer readable media include magnetic storage media (such as floppy disks, magnetic tapes, hard disk drives, etc.), optical magnetic storage media (e.g. magneto-optical disks), CD-ROM (compact disc read only memory), CD-R (compact disc recordable), CD-R/W (compact disc rewritable), DVD (Digital Versatile Disc), BD (Blu-ray (registered trademark) Disc), and semiconductor memories (such as mask ROM, PROM (Programmable ROM), EPROM (Erasable PROM), flash ROM, RAM (Random Access Memory), etc.). The program may be provided to a computer using any type of transitory computer readable media. Examples of transitory computer readable media include electric signals, optical signals, and electromagnetic waves. Transitory computer readable media can provide the program to a computer via a wired communication line (e.g. electric wires, and optical fibers) or a wireless communication line.

Part of or all the foregoing embodiments can be described as in the following appendixes, but the present invention is not limited thereto.

### (Supplementary Note 1)

An estimation apparatus comprising:

a first estimation unit configured to estimate a first pulse rate from a first video data which is captured a body part where a skin is exposed and output a first feature data derived based on the first video data in order to estimate the first pulse rate;

a training unit configured to train a determination model to determine a confidence value which indicates an reliability in the estimation of the first pulse rate based on the first feature data and a physiological information measured from the body;

an acquiring unit configured to acquire a second feature data derived by the first estimation unit when the first estimation unit estimates a second pulse rate from a second video data which is captured the body part to be estimated, and acquire the confidence value of the second pulse rate using the second feature data and the determination model trained by the training unit; and

a second estimation unit configured to estimate a third pulse rate based on the acquired confidence value.

### (Supplementary Note 2)

The estimation apparatus according to Supplementary Note 1, further comprising:

a feature data processing unit configured to output a third feature data by performing a predetermined statistic process to reduce noise for the first feature data; and

wherein the training unit trains the determination model using the third feature data as input of the determination model; and

wherein the acquiring unit acquires the confidence value of the second pulse rate using a fourth feature data as input of the determination model trained by the training unit, the fourth feature data is output by the feature data processing unit from the second feature data.

### (Supplementary Note 3)

The estimation apparatus according to Supplementary Note 2, wherein

the feature data processing unit performs at least one of color space transforms, a combination of filters and signal decomposition on the first feature data as the predetermined statistic process.

### (Supplementary Note 4)

The estimation apparatus according to any one of Supplementary Notes 1 to 3, wherein

the second estimation unit selects a reference periodicity information which is a reference of a period for each frame in the second video data based on the acquired confidence value and estimates the third pulse rate using the reference periodicity information.

### (Supplementary Note 5)

The estimation apparatus according to Supplementary Note 4, wherein

the second estimation unit estimates the third pulse rate by extracting, using the reference periodicity information, at least one of certain periodic components from multicomponent pulse signal which is extracted by the first estimation unit from each frame in the second video data.

### (Supplementary Note 6)

The estimation apparatus according to any one of Supplementary Notes 1 to 5, wherein

the physiological information is a measurement value which is a pulse rate measured from the body during capturing the first video data; and

the training unit trains the determination model so that the confidence value is determined to be higher as the first pulse rate is closer to the measurement value.

### (Supplementary Note 7)

The estimation apparatus according to any one of Supplementary Notes 1 to 6,

wherein the first estimation unit

detects feature points which configure the body part from each frame in the first video data,

identifies noise source of each frame based on the feature points,

selects ROI(a Region(s) Of Interest)s from each frame based on the feature points,

divides a plurality of sub regions from the ROIs,

extracts pulse signals from each of the plurality of sub regions,

generates ROI filters for each frame, each ROI filter is assigned each sub region with weights according to the identified noise source, and

estimates the first pulse rate by applying the extracted pulse signal to the ROI filters and performing frequency analysis on the filtered pulse signal.

### (Supplementary Note 8)

The estimation apparatus according to Supplementary Note 7, wherein

the first feature data includes at least one of the estimated first pulse rate, the detected feature points, the extracted pulse signal, the identified noise source, coefficients of the generated ROI filters and results of the frequency analysis.

### (Supplementary Note 9)

An estimation method using a computer comprising:

performing a first estimation process to estimate a first pulse rate from a first video data which is captured a body part where a skin is exposed;

outputting a first feature data derived based on the first video data in the first estimation process;

training a determination model to determine a confidence value which indicates a reliability in the estimation of the first pulse rate based on the first feature data and a physiological information measured from the body;

performing a second estimation process to estimate a second pulse rate from a second video data which is captured the body part to be estimated;

outputting a second feature data derived based on the second video data in the second estimation process;

acquiring the confidence value of the second pulse rate using the second feature data and the trained determination model; and

estimating a third pulse rate based on the acquired confidence value.

### (Supplementary Note 10)

A non-transitory computer readable medium storing a estimation program causing a computer to execute:

a first estimation process for estimating a first pulse rate from a first video data which is captured a body part where a skin is exposed;

a process for outputting a first feature data derived based on the first video data in the first estimation process;

a process for training a determination model to determine a confidence value which indicates a reliability in the estimation of the first pulse rate based on the first feature data and a physiological information measured from the body;

a second estimation process for estimating a second pulse rate from a second video data which is captured the body part to be estimated;

a process for outputting a second feature data derived based on the second video data in the second estimation process;

a process for acquiring the confidence value of the second pulse rate using the second feature data and the trained determination model; and

a process for estimating a third pulse rate based on the acquired confidence value.

### INDUSTRIAL APPLICABILITY

The present disclosure is applicable to a system and an apparatus for estimating physiological information aimed at stress detection, health care, and accident prevention.

### REFERENCE SIGNS LIST

100 PULSE RATE ESTIMATION APPARATUS
101 VIDEO CAPTURING UNIT
102 BODY PART TRACKING UNIT
103 NOISE SOURCE DETECTOR
104 ROI SELECTION AND PULSE EXTRACTION UNIT
105 PULSE RATE ESTIMATION UNIT
106 FEATURE SELECTOR
107 CONFIDENCE REGRESSOR
108 CONFIDENCE ESTIMATOR
109 REFERENCE SELECTOR
110 PERIODIC COMPONENT EXTRACTION UNIT
200 CAPTURED IMAGES
201 CAPTURED IMAGE
202 FEATURE POINTS
203 NOISE SOURCE LABELS
204 ROI SUB REGIONS
205 EXTRACTED PULSE SIGNAL
206 ROI FILTER
207 FILTERED PULSE SIGNAL
208 INITIAL ESTIMATED PULSE RATE
210 LOW LEVEL FEATURES
211 HIGH LEVEL FEATURES
212 TRUE PULSE RATE
213 CONFIDENCE LEVEL LABELS
214 TRAINED DETERMINATION MODEL
221 CAPTURED IMAGE
222 FEATURE POINTS
223 NOISE SOURCE LABELS
225 EXTRACTED PULSE SIGNAL
228 INITIAL ESTIMATED PULSE RATE
230 LOW LEVEL FEATURES
231 HIGH LEVEL FEATURES
232 CONFIDENCE LEVEL
233 REFERENCE PULSE RATE
234 PERIODIC COMPONENT
235 FINAL PULSE RATE
10 COMPUTER
11 CPU
12 MAIN MEMORY
13 STORAGE DEVICE
14 INPUT INTERFACE
15 DISPLAY CONTROLLER
16 DATA READER/WRITER
17 COMMUNICATION INTERFACE
18 INPUT DEVICE
19 DISPLAY DEVICE
20 RECORDING MEDIUM
21 BUS
30 ESTIMATION APPARATUS
31 FIRST ESTIMATION UNIT
32 TRAINING UNIT
33 ACQUIRING UNIT
34 SECOND ESTIMATION UNIT

What is claimed is:

1. An estimation apparatus comprising:
at least one memory storing instructions, and
at least one processor configured to execute the instructions to:
estimate a first pulse rate from a first video data which is captured a body part where a skin is exposed and output a first feature data derived based on the first video data in order to estimate the first pulse rate;
train a determination model to determine a confidence value which indicates reliability in the estimation of the first pulse rate based on the first feature data and a physiological information measured from the body;
acquire a second feature data derived by the first estimation unit when the first estimation unit estimates a second pulse rate from a second video data which is captured the body part to be estimated, and acquire the confidence value of the second pulse rate using the second feature data and the determination model trained by the training unit; and
estimate a third pulse rate based on the acquired confidence value.

2. The estimation apparatus according to claim 1,
wherein the at least one processor further configured to execute the instructions to
output a third feature data by performing a predetermined statistic process to reduce noise for the first feature data; and
train the determination model using the third feature data as input of the determination model; and
acquire the confidence value of the second pulse rate using a fourth feature data as input of the determination model trained by the training unit, the fourth feature data is output by the feature data processing unit from the second feature data.

3. The estimation apparatus according to claim 2,
wherein the at least one processor further configured to execute the instructions to
perform at least one of color space transforms, a combination of filters and signal decomposition on the first feature data as the predetermined statistic process.

4. The estimation apparatus according to claim 1, wherein the at least one processor further configured to execute the instructions to
select a reference periodicity information which is a reference of a period for each frame in the second video data based on the acquired confidence value and estimates the third pulse rate using the reference periodicity information.

5. The estimation apparatus according to claim 4, wherein the at least one processor further configured to execute the instructions to
estimate the third pulse rate by extracting, using the reference periodicity information, at least one of certain periodic components from multicomponent pulse signal which is extracted by the first estimation unit from each frame in the second video data.

6. The estimation apparatus according to claim 1, wherein
the physiological information is a measurement value which is a pulse rate measured from the body during capturing the first video data; and
wherein the at least one processor further configured to execute the instructions to
train the determination model so that the confidence value is determined to be higher as the first pulse rate is closer to the measurement value.

7. The estimation apparatus according to claim 1,
wherein the at least one processor further configured to execute the instructions to
detect feature points which configure the body part from each frame in the first video data,
identify noise source of each frame based on the feature points,
select ROI(a Region(s) Of Interest)s from each frame based on the feature points,
divide a plurality of sub regions from the ROIs,
extract pulse signals from each of the plurality of sub regions,
generate ROI filters for each frame, each ROI filter is assigned each sub region with weights according to the identified noise source, and
estimate the first pulse rate by applying the extracted pulse signal to the ROI filters and performing frequency analysis on the filtered pulse signal.

8. The estimation apparatus according to claim 7, wherein
the first feature data includes at least one of the estimated first pulse rate, the detected feature points, the extracted pulse signal, the identified noise source, coefficients of the generated ROI filters and results of the frequency analysis.

9. An estimation method using a computer comprising:
performing a first estimation process to estimate a first pulse rate from a first video data which is captured a body part where a skin is exposed;
outputting a first feature data derived based on the first video data in the first estimation process;
training a determination model to determine a confidence value which indicates a reliability in the estimation of the first pulse rate based on the first feature data and a physiological information measured from the body;
performing a second estimation process to estimate a second pulse rate from a second video data which is captured the body part to be estimated;
outputting a second feature data derived based on the second video data in the second estimation process;
acquiring the confidence value of the second pulse rate using the second feature data and the trained determination model; and
estimating a third pulse rate based on the acquired confidence value.

10. A non-transitory computer readable medium storing a estimation program causing a computer to execute:
a first estimation process for estimating a first pulse rate from a first video data which is captured a body part where a skin is exposed;
a process for outputting a first feature data derived based on the first video data in the first estimation process;
a process for training a determination model to determine a confidence value which indicates a reliability in the estimation of the first pulse rate based on the first feature data and a physiological information measured from the body;
a second estimation process for estimating a second pulse rate from a second video data which is captured the body part to be estimated;
a process for outputting a second feature data derived based on the second video data in the second estimation process;
a process for acquiring the confidence value of the second pulse rate using the second feature data and the trained determination model; and
a process for estimating a third pulse rate based on the acquired confidence value.
